(19) United States
(12) Patent Application Publication
(10) Pub. No.: US 2024/0185987 A1
(43) Pub. Date: Jun. 6, 2024

(54) APPARATUSES, SYSTEMS, AND METHODS FOR BIOMARKER COLLECTION, BI-DIRECTIONAL PATIENT COMMUNICATION AND LONGITUDINAL PATIENT FOLLOW-UP

(71) Applicant: Compass Pathfinder Limited, Altrincham, Cheshire (GB)

(72) Inventors: Jacob Birk, San Francisco, CA (US); Robert F. Dougherty, Redwood City, CA (US); Kwan Lye, San Francisco, CA (US); Yuchen Jiang, San Jose, CA (US); Dina Al-Alami, London (GB); Gregory A. Ryslik, Westerville, OH (US)

(21) Appl. No.: 17/927,462

(22) PCT Filed: Nov. 22, 2022
(86) PCT No.: PCT/US2022/050752

§ 371 Date: Nov. 23, 2022

Related U.S. Application Data
(60) Provisional application No. 63/282,633, filed on Nov. 23, 2021.

(51) Int. Cl. G16H 20/70; G16H 10/60; G16H 15/00; G16H 50/30

(52) U.S. Cl. CPC G16H 20/70; G16H 10/60; G16H 15/00; G16H 50/30

(57) ABSTRACT

Approaches for generating a set of biomarkers and generating a summary and one or more recommendations based, at least in part, upon the biomarkers are provided. Sensor data associated with a user may be received. The sensor data may be analyzed to generate a set of biomarkers. One or more biomarkers of the set may be combined to generate one or more additional biomarkers. A summary and one or more recommendations may be generated based, at least in part, upon the generated set of biomarkers and the additional biomarkers. The generated summary and the one or more recommendations may be provided for display on a client device.

### CROSS REFERENCE TO RELATED APPLICATIONS

This application is a 371 National Phase application from PCT Application No. PCT/US22/50752, filed on Nov. 22, 2022, and entitled “APPARATUSES, SYSTEMS, AND METHODS FOR BIOMARKER COLLECTION, BI-DIRECTIONAL PATIENT COMMUNICATION AND LONGITUDINAL PATIENT FOLLOW-UP,” which claims priority to U.S. Provisional Patent Application No. 63/282,633 filed Nov. 23, 2021, and entitled “APPARATUSES, SYSTEMS, AND METHODS FOR BIOMARKER COLLECTION, BI-DIRECTIONAL PATIENT COMMUNICATION, AND LONGITUDINAL PATIENT FOLLOW-UP,” which is hereby incorporated by reference herein in its entirety for all purposes.

### BACKGROUND

Today, many medical treatment and therapies are conducted with the aid of personal computers and smart devices. However, there is difficulty in correlating and properly utilizing patient biomarker data when communicating with patients. For example, biomarkers may be collected from a variety of devices, but often the data from such devices is high-level and may not provide detailed insights or recommendations for addressing changes from baseline scores. Further, such biomarker data may not be presented in a meaningful way, such that a user may be able to make changes to improve scores associated with the biomarker data.

### BRIEF DESCRIPTION OF THE DRAWINGS

Various embodiments in accordance with the present disclosure will be described with reference to the drawings, in which:

FIG. 1 illustrates an example interface that can be used to implement aspects of the various embodiments.

FIG. 2 illustrates another example interface that can be used to implement aspects of the various embodiments.

FIG. 3 illustrates an example system that can be utilized to implement one or more aspects of the various embodiments.

FIG. 4 illustrates an example method that can be utilized to implement one or more aspects of the various embodiments.

FIG. 5 illustrates an example of an environment for implementing one or more aspects of the various embodiments.

FIG. 6 illustrates an example block diagram of an electronic device that can be utilized to implement one or more aspects of the various embodiments.

FIG. 7 illustrates components of another example environment in which aspects of various embodiments can be implemented.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Approaches for generating a set of biomarkers and generating a summary and one or more recommendations based, at least in part, upon the biomarkers are provided. Sensor data associated with a user may be received. The sensor data may be analyzed to generate a set of biomarkers. One or more biomarkers of the set may be combined to generate one or more additional biomarkers. A summary and one or more recommendations may be generated based, at least in part, upon the generated set of biomarkers and the additional biomarkers. The generated summary and the one or more recommendations may be provided for display on a client device.

In accordance with an example embodiment, a data-secure and anonymized cloud-based platform for biomarker collection, bi-directional patient communication, and longitudinal patient follow-up may be provided. A cloud-based confidential architecture for digital biomarker collection may be utilized in digital biomarker collection, using smartphone sensors, wearables, and other such smart devices. Additionally, an anonymized, confidential, cloud-native patient communication system may be provided, enabling delivery of patient education material as well as multi-party communication between the patient and care team. Further, content may be delivered securely and synchronously to patients and care teams.

In accordance with one or more embodiments, a secure artificial intelligence biomarker action and response service configured to provide real-time patient and therapist feedback on predetermined triggers may be provided. As a non-limiting example, if a patient typically sleeps for eight hours during a given night after an initial dosing session of an administered therapy, and the patient only receives five hours of sleep per night several months after dosing, this sleep change may be an indication of patient relapse or health deterioration that may be flagged for a care team.

Information may be dynamically loaded from a cloud-based environment, enabling global synchronization of all devices in terms of content. Further, the system may enable content to be customized, such as by enabling artificial intelligence, machine learning, and/or the care team to make customizations prior to displaying the content. The customized content may be managed remotely or locally. Because customizations can be made in this way, different versions of the application may not have to be released for different studies or use cases. The application may be applicable across various studies and use cases using global synchronization. In some embodiments, at least a subset of data and information presented to a user is stored locally but updated from a remoted cloud-based backend. The application may dynamically retrieve information from the cloud-based backend and information may be downloaded on the fly. In accordance with an example embodiment, patients may be assigned to specific studies, and a cloud-based algorithm may perform an analysis of which information is relevant per patient. A global list of all required information may be created, and relevant content may be provided in real time or near-real time. If a connection to a cloud-based environment is not available, data from a last previous cloud pull may be shown. If there has never been a last previous cloud pull, default information may be shown until connection to the Internet is available. As such, the system may be able to provide different content for different patients and the same patient over time in a singular application.

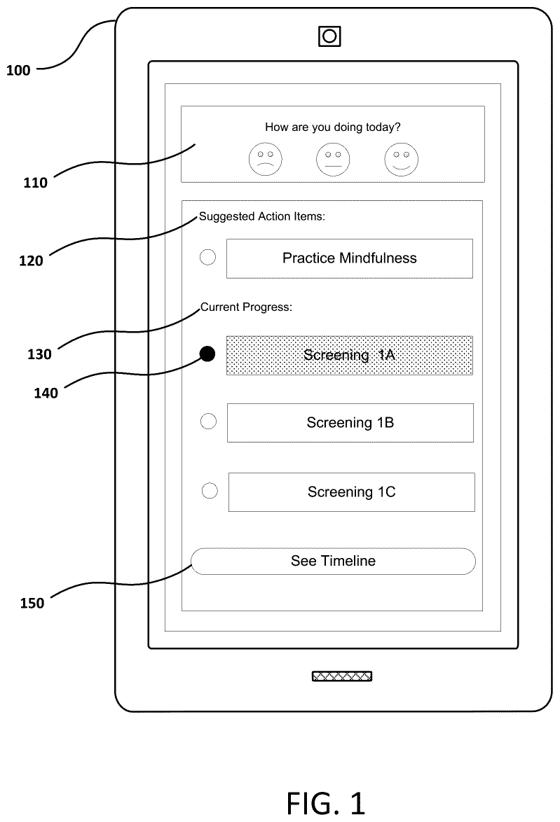

FIG. 1 illustrates an example interface 100 that can be used to implement aspects of the various embodiments. In accordance with an example embodiment, a client device may have an interface, which can display various elements. A client device may include a smart phone, tablet, or personal computer such as a laptop or desktop computer, among other such devices. An interface, in accordance with an example embodiment, may include elements such as a check-in 110, where a user can input or select a reaction corresponding to their mood at that point in time. Elements of the interface may also include suggested action items 120 and current progress tracking 140. Suggested action items 120 may include recommended actions a user should take. Recommended actions may be determined using machine learning or other artificial intelligence techniques, and may be determined using one or more biomarkers analyzed for a user. As a non-limiting example, biomarkers associated with a user may indicate that the user may be unfocused, anxious, or is not getting sufficient sleep. For example, one or more scores associated with the user's biomarkers may fall below a determined threshold. As a result, the system may determine that the user would benefit from practicing mindfulness. The system may present the recommendation for practicing mindfulness as an action item for the user to take. The user may simply click on, tap, or otherwise interact with the recommended action item, and a course for mindfulness may be loaded for the user to partake in.

In accordance with another example embodiment, a user may be presented with the current progress 130 of their therapeutic journey. The current progress may include mandatory screenings a user is required to complete before a given date. For example, a user may be required to complete Screenings 1A-1C. As a user completes the individual items, completed items may be presented in a manner that indicates that the item does not require further action. For example, Screening 1A (corresponding to element 140) has been completed and is therefore being displayed as being grayed out, with a darkened circle next to the item. If a user taps on, clicks on, or otherwise interacts with the items, the user may be taken to an electronic page providing content for the item. If a user would like to track their progress so far, they may tap on, click on, or otherwise interact with an element to see a timeline, such as element 150. In accordance with an example embodiment, the timeline may display completed action items in chronological order.

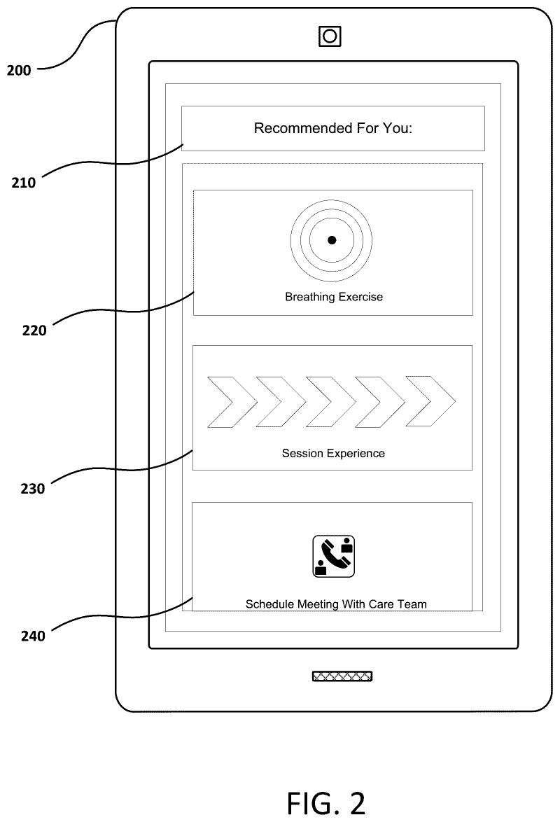

FIG. 2 illustrates another example interface 200 that can be used to implement aspects of the various embodiments. In accordance with an example embodiment, an interface 200 may present various recommendations 210 for a user to consider. In some embodiments, the recommendations may be tailored to the specific user using artificial intelligence techniques. In other embodiments, the recommendations may include predetermined recommendations that may be generally applicable to a set of users. In some embodiments, the recommendations may be a combination of tailored recommendations and generally-applicable recommendations.

In this example embodiment, recommendations may include breathing exercises 220, session experience information 230, and an option to schedule a meeting with a care team 240. Breathing exercises may include information about breathing exercises, along with resources for guided meditations that a user may be able to use. Session experience information may include general information related to the therapy that the user is undergoing. Session experience information may also include information related to the user's specific therapeutic experience. The user, in some example embodiments, may provide feedback about their subjective experience while undergoing a therapy. A user may also, in accordance with an example embodiment, be able to schedule a meeting with a care team using element 240. Interacting with the element may cause a virtual scheduling assistant to open, where a user may select a date and time that works for them, and may specify if they would like a video call, phone call, or text chat. If there are no windows of time that work for the user, the user may be able to leave a video, phone, or text message for their care team.

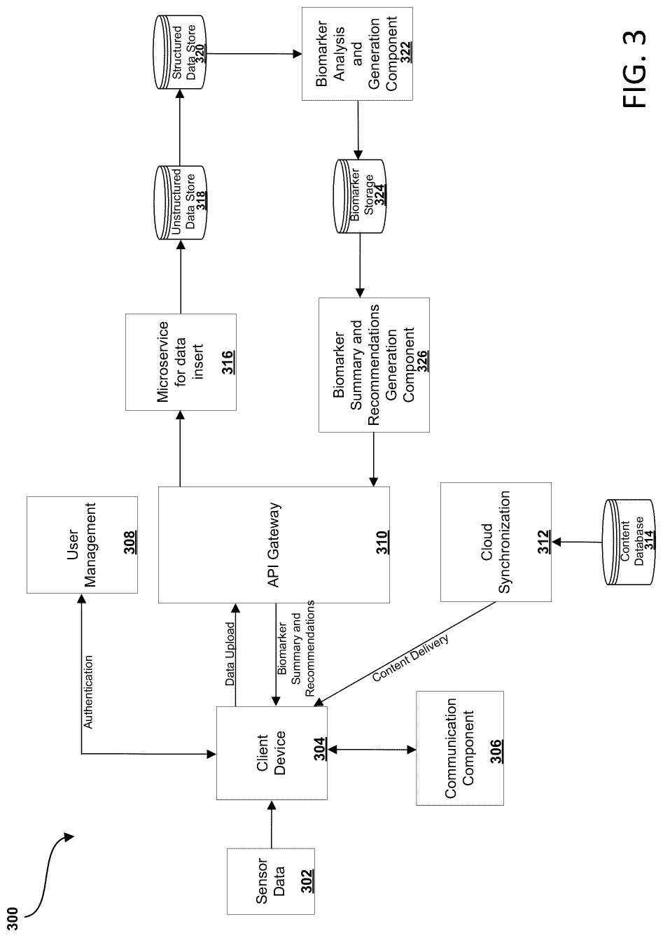

FIG. 3 illustrates an example system 300 that can be utilized to implement one or more aspects of the various embodiments. The system, in accordance with an example embodiment, may include a mobile application or a web-based application, a secure layer, a data processing layer, and/or a patient feedback layer. The system may include a fully secure and anonymized cloud-based platform for biomarker collection, bi-directional patient communication, and/or longitudinal patient follow-up.

A patient may grant permission for biomarker collection, including collection of sensor data. In accordance with an example embodiment, sensor data 320 may be provided to or collected by a client device 304. Sensor data may be collected at various time points using a variety of sensors, including, but not limited to, sensors of a smartphone, sensors of wearable devices, and sensors of imaging devices. Sensors, in accordance with an example embodiment, may include, but are not limited to, light sensors, global positioning system (GPS) sensors, accelerometers, gyroscopes, magnetometers, barometers, network connectivity sensors, activity state sensors, screen touch events, data from paired wearables (e.g., heart rate sensors, glucose levels, neck wearables detecting brain activity, etc.), and other such sensors and signals. In accordance with an example embodiment, personal identifying information associated with sensor data may be masked to maintain a user's privacy. A client device may include, but is not limited to, a smart phone, tablet, personal computing device, or wearable device, among other such devices. A user management module 308 may be utilized, in accordance with an example embodiment, to authenticate one or more client devices. In some embodiments, patients may have their own user accounts under a unique identifier. The user account may contain a subject identifier, such as an indication as to where a patient is in their therapeutic journey. In another embodiment, the user account may contain more extensive information, such as whether the patient is part of an affiliated program. The subject identifier may serve as a global identifier for all communications, preparation, biomarker collection, or other interaction, and may uniquely identify a user while preserving anonymity of the actual individual. Each subject identifier may be computer generated and assigned to the individual. In other embodiments, an administrator may assign a subject identifier to a patient.

In at least one example embodiment, the system may enable access to a patient or care team via a mobile application or web-based application. When a user account is created, the user may be able to log in, at which point the application will download the latest preparation materials that the user is authenticated for. As a non-limiting example, the application may download pre-reading material for an upcoming treatment session.

A communication component, such as communication component 306, may be utilized to connect a client device with a member of a care team. Communication component 306 may support video conferencing, teleconferencing, and text-based communications between users. If user permissions allow, a patient and care team may be able to communicate directly with each other, such as through video, text, and/or audio, both synchronously and asynchronously. For example, a patient may leave a message for their care team if someone is not available to speak on demand, or a patient may be able to speak with a member of their care team in real time. In this way, a patient may be able to prepare for an upcoming therapy session. Additionally, a patient may be able to follow up with their care team before and/or after the treatment session as needed. The application, in accordance with an example embodiment, may allow capture of patient and care team communication for later processing. For example, communications may be processed using natural language processing and/or other artificial intelligence analysis as appropriate and allowed by patient consent. In one example embodiment, even if only the subject identifier is available, anomaly detection may allow unsupervised machine learning algorithms to detect deviations from a baseline measurement. Specifically, in some example embodiments, Bayesian analysis may be used to combine population-level measurements with per-patient specific data to determine a baseline per patient. Sustained deviation from this baseline may represent a substantial deviation from that patient's healthy state. For example, if the average population level sleep amount is eight hours per night, but the patient typically sleeps six hours per night, a normal range may be created for a patient between 5.75-6.5 hours per night. However, if the patient begins to consistently only receive four hours of sleep per night, this may represent a statistically and medically significant deviation from the patient's normal range.

A content database 314 may store content to be provided for display on a client device through a cloud synchronization module 312. Content database may include educational content which may help assist a user through their therapeutic journey. While this example uses a cloud synchronization module, content, in other example embodiments, may be stored locally on the client device. The application may provide anonymized data collection, educational content, biomarker summary, and/or suggestions generated using artificial intelligence. Sensor data may be uploaded in a secure layer to an application programming interface (API) Gateway 310, including secure entry to backend services and logic, via client device 304. Referring to the secure layer, data sent to the cloud based environment, from the application, may proceed through a secure layer. A secure layer, in accordance with an example embodiment, may include an API gateway.

Data may be sent, from API gateway 310, to a data processing layer that may structure data, provide machine learning biomarker creation, and generate suggestions or recommendations using artificial intelligence based on one or more biomarkers. The system may trigger a function, such as a microservice for data insert 316, configured to handle database inserts. In accordance with an example embodiment, biomarker collection may be feature flagged, with individual sensors and study settings being toggled based on patient preferences. Data may be injected dynamically, for example, via a serverless lambda function that may be configured to rectify missing data. However, data may be injected via any suitable means.

In an example embodiment, each request and response may be validated by the API gateway that serves as an entry-point to the backend. The API gateway may ensure requests are authenticated before sending requests to backend services and databases. Further, non-personalized educational content may be synchronized across mobile applications.

Referring now to the data processing layer, collected patient data may be inserted as semi-structured or unstructured data into a database (e.g., a NoSQL database), such as data store 318. Further, the data may be processed and inserted into a structured data warehouse, such as structured data store 320. Such processing may occur on a regular basis or at pre-determined intervals of time. The structured data may be analyzed, where digital biomarkers may be generated for each patient, using biomarker analysis and generation component 322. In accordance with an example embodiment, the biomarkers may be processed with artificial intelligence to identify deviations. Input from a plurality of sensors may be processed using a machine learning framework or unsupervised framework to help analyze the data and generate one or more conclusions from the data. In accordance with an example embodiment, any number of the sensors may be configured to generate novel measurements of an individual that would otherwise require additional hardware. For example, input from the light sensor may be combined with accelerometer data to generate a measurement of patient sleep that can then be used to understand the patient's health at that point in time. Light sensor data and accelerometer data, standing alone, may not be as accurate as the combination of data. The overall integration of any number the sensors into a single machine learning algorithm may enable the possibility to continuously monitor patient health and outcomes of interest. In one example embodiment, the biomarkers may be processed in a cloud-based environment. In other example embodiments, the biomarkers may be processed locally or on any suitable computing device. The processed biomarkers may be transmitted to an application, such as a mobile application or a web-based application, for a care team to review. Biomarkers may be transmitted at any time, such as in real time, near-real time, or at a determined point in time. A trigger may alert a care team if a threshold has been met with respect to one or more biomarkers. For example, if a patient has a sleep range of 7-8 hours per night at a 95% confidence level, the system may alert a care team if the patient sleeps 4-5 hours per night at a 95% confidence level, as this decrease in sleep may be a signal of mental health deterioration. Thresholds may be patient-specific, and may be generated using a model where individual patients have their own individual baseline. Baselines may be combined with an average baseline (e.g., an average baseline for a given population), to generate an estimation with relevance. In an example embodiment, a Bayesian model may be utilized to generate the estimation.

Biomarkers may be stored, such as in biomarker storage 324. Biomarker data may be stored temporally, such that the data may be timestamped. In this way, a patient's progress throughout therapy may be more easily tracked over time.

A patient feedback layer may provide biomarker summaries, suggestions on content and other action items, and/or real-time patient-professional communication. Biomarker processing may help generate human-readable summaries and recommendations, which may become available for the patient in an application. Summaries and recommendations may be generated at biomarker summary and recommendations generation component 326, in an example embodiment. Summaries and recommendations may depend on the biomarker and patient, but such information may provide the patient's historical level, any deviations outside the norm (e.g., outside of the patient's 95% confidence bounds), and potential suggestions if a pattern is determined. The summaries and/or recommendations may also provide information depicting why the summary is useful and/or reflective of patient health. As a non-limiting example, if the system determines that sleep is more restful on days when the patient takes a walk of at least thirty minutes, a suggestion might be provided to the application if the patient has not taken their walk by 5 PM of that day. Accordingly, such a summary may display the patient's historical sleep over the last several months, along with deviations outside of the norm. As a function of recommendations generated using artificial intelligence, the system may send notifications to the patient for action items.

Referring to the patient feedback layer, a biomarker summary may be presented to the patient together with suggested action items and recommended content. In an example embodiment, as a function of the suggested action items, a real-time video chat may be scheduled with an assigned therapist or other member of the care team. Chats may follow any suitable schedule (e.g., twice a week), depending on the patient's progress in treatment. Surveys, such as short-structured surveys, may be presented to the patient that may be used for further enhancements of the biomarkers. As a non-limiting example, surveys may be used to directly assess patient mental health and patient status. In various embodiments, the system may recreate or provide similar assessments to common mental health assessments such as, but not limited to, the Montgomery-Asberg Depression Rating Scale (MADRS), a patient health questionnaire, or 9-item (PHQ-9) that may be used clinically. Alternatively, surveys may include a subjective element or may reflect questions that a user feels to be more appropriate to measure patient health. For example, such a question may include “how many times has the subject engaged in physical activity over the last seven days?” In accordance with an example embodiment, the results of these surveys may be used as training labels for supervised machine learning.

The application may be configured to recommend content to the user. For example, the recommendations may be presented to the user in an application under a tab. The recommendations may be generated, in accordance with an example embodiment, based on data collected from the patient. For example, if the user's collected data shows elevated anxiety levels, the user may be recommended to watch a breathing exercise video to promote relaxation.

Additionally, a user may be asked how they are doing, and be provided with options to reply based on their contentment. In accordance with an example embodiment, replies may be stored and considered as part of the biomarker analysis. The user may also be presented with a schedule of upcoming sessions and a history of past sessions.

In the application, the user may also be directed to video, audio, or text chats with a member of their care team. The user may also be prompted to provide personal details or adjust settings. A member of a care team or administrator may be provided access to a backend of the application.

Various means of authentication may be provided, in accordance with an example embodiment. For example, a login portal may be provided, to enable anonymity of the patient and care team. Further, the system may include automated cross-platform digital biomarker and sensor data collection, such as across various operating systems, that may be integrated into a single cloud-native database. Additionally, the system may include a synchronized content database that is remotely updated for new studies and patients. In at least some example embodiments, a user may utilize a quick response (QR) code to log into one or more features of the service environment.

In one example embodiment, the system may be a cross-platform front-end application that is consistent across all devices, enabling for easier validation of the application for regulatory purposes as one codebase. Moreover, the application may allow for the display of custom biomarkers to the care team and patient. Further, the application may provide custom alerting as a function of the data collected. The application may allow for “full circle” machine learning, as the system may be configured to collect data, upload, analyze, identify triggers, and/or transmit information to the care team.

The system may further be configured to enable a patient to sign up for treatment, agree to consent forms, and/or download a mobile application or access a web-based application. The patient may sign in to the application using a secure and encrypted login process. During an onboarding process, the patient may allow access to data collection and notifications (e.g., the patient may grant permission via a mobile device). In an example embodiment, the application may collect health data, movement data, data from external wearables, and device usage data when available. Collected data may be encrypted, anonymized, and uploaded to a cloud-based environment. The application may also suggest educational content for the patient to browse and consume, such as text, video, and/or audio, as a function of the patient's process through the treatment process.

The application may include an administrator user interface for managing studies, treatments, and/or participants. The administrator user interface may be presented to an administrator via a mobile device or other computerized device. Such a user interface may permit the administrator to add or remove participants to one or more studies and/or locations. For example, the one or more studies and/or one or more locations may populate one or more drop down menus. Further, such a user interface may present the administrator with the identifier of the participant and the time and date when it was created. The user interface may also present the administrator with the ability to expand the participant's identifier and view more details about the participant and/or study.

The application may present the participant with a one-time login code and/or QR code to access the mobile application. For example, such a code may be presented to the participant upon the outset of treatment. In an example embodiment, the administrator and/or the user may cause the system to generate a new code. Each login code and/or QR code may be associated with a unique subject identifier.

The application may also include one or more permissions, which may be enabled or disabled in accordance with one or more embodiments. Permissions may include, but are not limited to, active energy, electrodermal activity, flights climbed, heart rate, heart rate variability, mindful minutes, oxygen saturation, resting heart rate, sleep, steps, walking and running distance, and walking heart rate average. The user may prevent the application from utilizing the corresponding type of data or sensor.

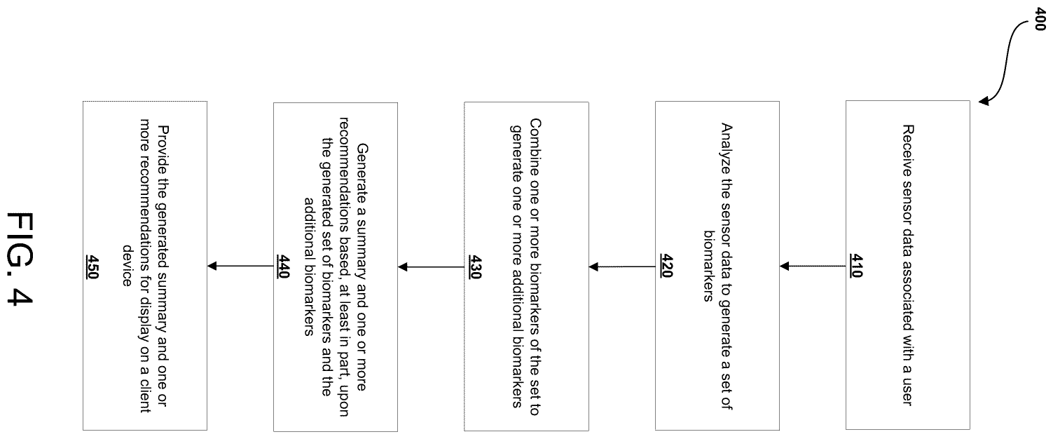

FIG. 4 illustrates an example method that can be utilized to implement one or more aspects of the various embodiments. It should be understood that for any process herein there can be additional, fewer, or alternative steps performed in similar or alternative orders, or in parallel, within the scope of the various embodiments unless otherwise specifically stated. In accordance with an example embodiment, sensor data associated with a user may be received 410. The sensor data may be analyzed to generate a set of biomarkers 420. One or more biomarkers of the set may be combined to generate one or more additional biomarkers 430. A summary and recommendations related to the biomarkers may be generated based, at least in part, upon the generated set of biomarkers and the additional biomarkers 440. The generated summary can be provided for display on a client device 450.

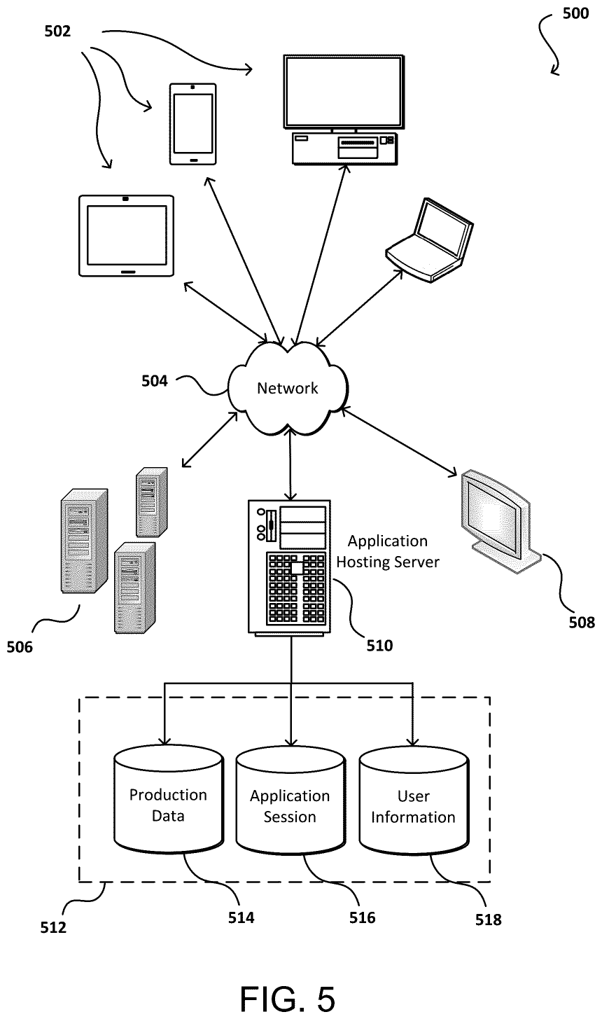

As discussed, different approaches can be implemented in various environments in accordance with the described embodiments. For example, FIG. 5 illustrates an example of an environment 500 for implementing one or more aspects of the various embodiments. As will be appreciated, although a Web-based environment is used for purposes of explanation, different environments may be used, as appropriate, to implement various embodiments. The system includes an electronic client device 502, 508, which can include any appropriate device operable to send and receive requests, messages or information over an appropriate network 504 and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes one or more servers 506 for receiving requests and serving content in response thereto, although for other networks, an alternative device serving a similar purpose could be used, as would be apparent to one of ordinary skill in the art.

The illustrative environment includes at least one application server 510 and a data store 512. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 510 can include any appropriate hardware and software for integrating with the data store 512 as needed to execute aspects of one or more applications for the client device and handling a majority of the data access and business logic for an application. The application server provides access control services in cooperation with the data store and is able to generate content such as text, graphics, audio and/or video to be transferred to the user, which may be served to the user by the one or more servers 506, including a Web server, in the form of HTML, XML or another appropriate structured language in this example. The handling of all requests and responses, as well as the delivery of content between the client device 502, 508 and the application server 510, can be handled by the Web server of servers 506. It should be understood that the Web and application servers are not required and are merely example components, as structured code discussed herein can be executed on any appropriate device or host machine as discussed elsewhere herein.

The data store 512 can include several separate data tables, databases or other data storage mechanisms and media for storing data relating to a particular aspect. For example, the data store illustrated includes mechanisms for storing production data 514 and user information 518, which can be used to serve content for the production side. The data store is also shown to include a mechanism for storing log or application session data 516. It should be understood that there can be many other aspects that may need to be stored in the data store, such as page image information and access rights information, which can be stored in any of the above listed mechanisms as appropriate or in additional mechanisms in the data store 512. The data store 512 is operable, through logic associated therewith, to receive instructions from the application server 510 and obtain, update or otherwise process data in response thereto. In one example, a user might submit a request for transcribing, tagging, and/or labeling a media file. In this case, the data store might access the user information to verify the identity of the user and can provide a transcript including tags and/or labels along with analytics associated with the media file. The information can then be returned to the user, such as in a results listing on a Web page that the user is able to view via a browser on the user device 502, 508. Information for a particular feature of interest can be viewed in a dedicated page or window of the browser.

Each server typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 5. Thus, the depiction of the system 500 in FIG. 5 should be taken as being illustrative in nature and not limiting to the scope of the disclosure.

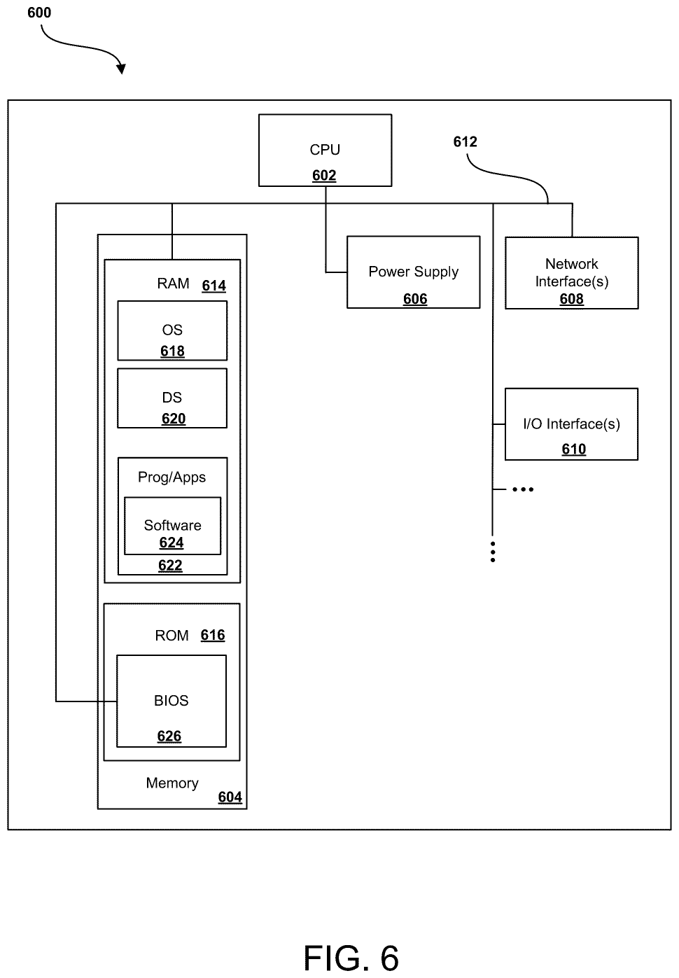

FIG. 6 illustrates an example block diagram of an electronic device that can be utilized to implement one or more aspects of the various embodiments. Instances of the electronic device 600 may include one or more servers and one or more client devices. In general, the electronic device may include a processor/CPU 602, memory 604, a power supply 606, and input/output (I/O) components/devices 610, e.g., microphones, speakers, displays, touchscreens, keyboards, mice, keypads, microscopes, GPS components, cameras, heart rate sensors, light sensors, accelerometers, targeted biometric sensors, neck wearables detecting brain activity, etc., which may be operable, for example, to provide graphical user interfaces or text user interfaces.

A user may provide input via a touchscreen of an electronic device 600. A touchscreen may determine whether a user is providing input by, for example, determining whether the user the user is touching the touchscreen with a part of the user's body such as their fingers. The electronic device 600 can also include a communications bus 612 that connects to the aforementioned elements of the electronic device 600. Network interfaces 608 can include a receiver and a transmitter (or a transceiver), and one or more antennas for wireless communications.

The processor 602 can include one or more of any type of processing device, e.g., a Central Processing Unit (CPU), and a Graphics Processing Unit (GPU). Also, for example, the processor can utilize central processing logic, or other logic, may include hardware, firmware, software or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 604, which can include Random Access Memory (RAM) 614 and Read Only Memory (ROM) 616, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 618, data storage 620, which may include one or more databases, and programs and/or applications 622, which can include, for example, software aspects of the program 624. The ROM 616 can also include Basic Input/Output System (BIOS) 626 of the electronic device 600.

Software aspects of the program 622 are intended to broadly include or represent all programming, applications, algorithms, models, software and other tools necessary to implement or facilitate methods and systems according to embodiments of the invention. The elements may exist on a single computer or be distributed among multiple computers, servers, devices, or entities.

The power supply 606 may contain one or more power components, and may help facilitate supply and management of power to the electronic device 600.

The input/output components, including Input/Output (I/O) interfaces 610, can include, for example, any interfaces for facilitating communication between any components of the electronic device 600, components of external devices, and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 610 and the bus 612 can facilitate communication between components of the electronic device 600, and in an example can ease processing performed by the processor 602.

Where the electronic device 600 is a server, it can include a computing device that can be capable of sending or receiving signals, e.g., a wired or wireless network, or may be capable of processing or storing signals, e.g., in memory as physical memory states. The server may be an application server that includes a configuration to provide one or more applications via a network to another device. Also, an application server may, for example, host a website that can provide a user interface for administration of example embodiments.

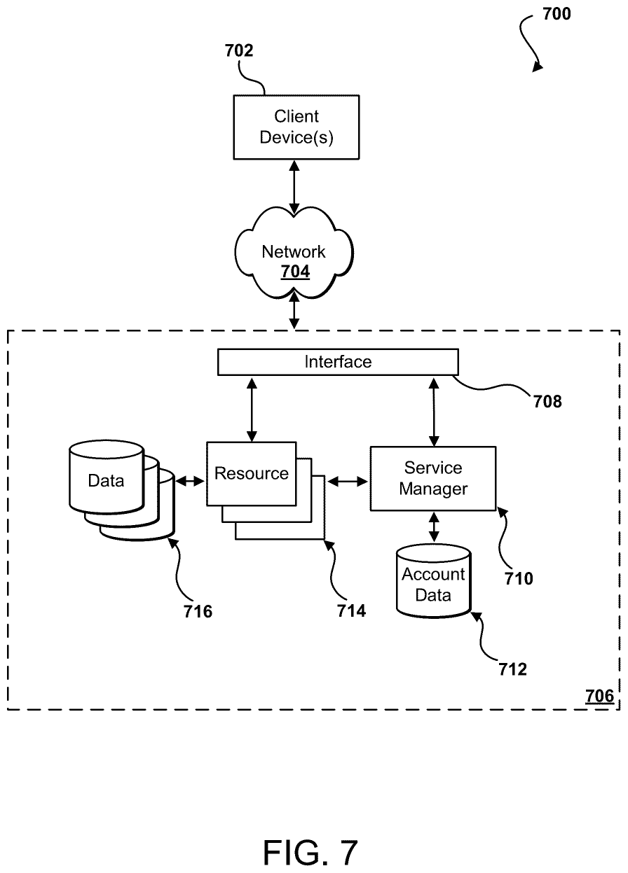

FIG. 7 illustrates an example environment 700 in which aspects of the various embodiments can be implemented. In this example a user is able to utilize one or more client devices 702 to submit requests across at least one network 704 to a multi-tenant resource provider environment 706. The client device can include any appropriate electronic device operable to send and receive requests, messages, or other such information over an appropriate network and convey information back to a user of the device. Examples of such client devices include personal computers, tablet computers, smart phones, notebook computers, and the like. The at least one network 704 can include any appropriate network, including an intranet, the Internet, a cellular network, a local area network (LAN), or any other such network or combination, and communication over the network can be enabled via wired and/or wireless connections. The resource provider environment 706 can include any appropriate components for receiving requests and returning information or performing actions in response to those requests. As an example, the provider environment might include Web servers and/or application servers for receiving and processing requests, then returning data, Web pages, video, audio, or other such content or information in response to the request.

In various embodiments, the provider environment may include various types of resources that can be utilized by multiple users for a variety of different purposes. As used herein, computing and other electronic resources utilized in a network environment can be referred to as “network resources.” These can include, for example, servers, databases, load balancers, routers, and the like, which can perform tasks such as to receive, transmit, and/or process data and/or executable instructions. In at least some embodiments, all or a portion of a given resource or set of resources might be allocated to a particular user or allocated for a particular task, for at least a determined period of time. The sharing of these multi-tenant resources from a provider environment is often referred to as resource sharing, Web services, or “cloud computing,” among other such terms and depending upon the specific environment and/or implementation. In this example the provider environment includes a plurality of resources 714 of one or more types. These types can include, for example, application servers operable to process instructions provided by a user or database servers operable to process data stored in one or more data stores 716 in response to a user request. As known for such purposes, the user can also reserve at least a portion of the data storage in a given data store. Methods for enabling a user to reserve various resources and resource instances are well known in the art, such that detailed description of the entire process, and explanation of all possible components, will not be discussed in detail herein.

In at least some embodiments, a user wanting to utilize a portion of the resources 714 can submit a request that is received to an interface layer 808 of the provider environment 706. The interface layer can include application programming interfaces (APIs) or other exposed interfaces enabling a user to submit requests to the provider environment. The interface layer 808 in this example can also include other components as well, such as at least one Web server, routing components, load balancers, and the like. When a request to provision a resource is received to the interface layer 708, information for the request can be directed to a service manager 710 or other such system, service, or component configured to manage user accounts and information, resource provisioning and usage, and other such aspects. A service manager 710 receiving the request can perform tasks such as to authenticate an identity of the user submitting the request, as well as to determine whether that user has an existing account with the resource provider, where the account data may be stored in at least one data store 712 in the provider environment. A user can provide any of various types of credentials in order to authenticate an identity of the user to the provider. These credentials can include, for example, a username and password pair, biometric data, a digital signature, a QR-based credential, or other such information.

The provider can validate this information against information stored for the user. If the user has an account with the appropriate permissions, status, etc., the resource manager can determine whether there are adequate resources available to suit the user's request, and if so can provision the resources or otherwise grant access to the corresponding portion of those resources for use by the user for an amount specified by the request. This amount can include, for example, capacity to process a single request or perform a single task, a specified period of time, or a recurring/renewable period, among other such values. If the user does not have a valid account with the provider, the user account does not enable access to the type of resources specified in the request, or another such reason is preventing the user from obtaining access to such resources, a communication can be sent to the user to enable the user to create or modify an account, or change the resources specified in the request, among other such options. In at least some example embodiments, a user may be authenticated to access an entire fleet of services provided within a service provider environment. In other example embodiments, a user's access may be restricted to specific services within the service provider environment using one or more access policies tied to the user's credential(s).

Once the user is authenticated, the account verified, and the resources allocated, the user can utilize the allocated resource(s) for the specified capacity, amount of data transfer, period of time, or other such value. In at least some embodiments, a user might provide a session token or other such credentials with subsequent requests in order to enable those requests to be processed on that user session. The user can receive a resource identifier, specific address, or other such information that can enable the client device 702 to communicate with an allocated resource without having to communicate with the service manager 710, at least until such time as a relevant aspect of the user account changes, the user is no longer granted access to the resource, or another such aspect changes.

The service manager 710 (or another such system or service) in this example can also function as a virtual layer of hardware and software components that handles control functions in addition to management actions, as may include provisioning, scaling, replication, etc. The resource manager can utilize dedicated APIs in the interface layer 808, where each API can be provided to receive requests for at least one specific action to be performed with respect to the data environment, such as to provision, scale, clone, or hibernate an instance. Upon receiving a request to one of the APIs, a Web services portion of the interface layer can parse or otherwise analyze the request to determine the steps or actions needed to act on or process the call. For example, a Web service call might be received that includes a request to create a data repository.

An interface layer 708 in at least one embodiment includes a scalable set of user-facing servers that can provide the various APIs and return the appropriate responses based on the API specifications. The interface layer also can include at least one API service layer that in one embodiment consists of stateless, replicated servers which process the externally-facing user APIs. The interface layer can be responsible for Web service front end features such as authenticating users based on credentials, authorizing the user, throttling user requests to the API servers, validating user input, and marshalling or unmarshalling requests and responses. The API layer also can be responsible for reading and writing database configuration data to/from the administration data store, in response to the API calls. In many embodiments, the Web services layer and/or API service layer will be the only externally visible component, or the only component that is visible to, and accessible by, users of the control service. The servers of the Web services layer can be stateless and scaled horizontally as known in the art. API servers, as well as the persistent data store, can be spread across multiple data centers in a region, for example, such that the servers are resilient to single data center failures.

The various embodiments can be further implemented in a wide variety of operating environments, which in some cases can include one or more user computers or computing devices which can be used to operate any of a number of applications. User or client devices can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network that would be familiar to those skilled in the art for supporting communications using any of a variety of commercially available protocols, such as TCP/IP, FTP, UPnP, NFS, and CIFS. The network can be, for example, a local area network, a wide-area network, a virtual private network, the Internet, an intranet, an extranet, a public switched telephone network, an infrared network, a wireless network and any combination thereof. In embodiments utilizing a Web server, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C# or C++ or any scripting language, such as Perl, Python or TCL, as well as combinations thereof. The server(s) may also include database servers, including without limitation those commercially available from Oracle®, Microsoft®, Sybase® and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, optical storage devices and solid-state storage devices such as random access memory (RAM) or read-only memory (ROM), as well as removable media devices, memory cards, flash cards, etc. Such devices can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information.

The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed. Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

What is claimed is:

1. A computer-implemented method, comprising:
receiving sensor data associated with a user;
analyzing the sensor data to generate a set of biomarkers;
combining one or more biomarkers of the set to generate one or more additional biomarkers;
generating a summary and one or more recommendations based, at least in part, upon the generated set of biomarkers and the additional biomarkers; and
providing the generated summary for display on a client device.

2. The computer-implemented method of claim 1, wherein the sensor data is analyzed using at least one neural network.

3. The computer-implemented method of claim 1, wherein the summary and the one or more recommendations are determined using machine learning.

4. The computer-implemented method of claim 1, wherein the sensor data is collected from one or more of: light sensors, global positioning system (GPS) sensors, accelerometers, gyroscopes, magnetometers, barometers, network connectivity sensors, activity state sensors, screen touch events, and data from paired wearables.

5. The computer-implemented method of claim 1, further comprising:
determining a score for at least one biomarker;
determining that the score falls below a determined threshold; and
generating the one or more recommendations based, at least in part, upon the score.

6. A computer-implemented method, comprising:
receiving first sensor data associated with a user;
analyzing the first sensor data to generate a set of biomarkers;
generating one or more recommendations based, at least in part, upon the generated set of biomarkers;
providing the one or more recommendations for display on a client device;
determining that the user has taken an action with respect to the one or more recommendations;
analyzing second sensor data associated with the user; and
causing an indication to be provided for display with respect to the analyzed second sensor data.

7. The computer-implemented method of claim 6, further comprising:
combining a plurality of biomarkers, of the set of biomarkers, to generate a new biomarker; and
generating the one or more recommendations based, at least in part, upon the generated set of biomarkers and the new biomarker.

8. The computer-implemented method of claim 6, wherein at least one of the first sensor data and the second sensor data is analyzed using at least one neural network.

9. The computer-implemented method of claim 6, wherein the one or more recommendations are determined using machine learning.

10. The computer-implemented method of claim 6, wherein the sensor data is collected from one or more of: light sensors, global positioning system (GPS) sensors, accelerometers, gyroscopes, magnetometers, barometers, network connectivity sensors, activity state sensors, screen touch events, and data from paired wearables.

11. The computer-implemented method of claim 6, further comprising:
determining a score for at least one biomarker;
determining that the score falls below a determined threshold; and
generating the one or more recommendations based, at least in part, upon the score.

12. The computer-implemented method of claim 6, further comprising:
determining a baseline score for one or more biomarkers of the set;
determining that a second score for the one or more biomarkers fall below the determined baseline score; and
generating the one or more recommendations based, at least in part, upon the baseline score and the second score.

13. The computer-implemented method of claim 6, further comprising:
determining a score for at least one biomarker;
generating an alert if the score falls below a determined threshold; and
providing the alert for display on the client device.

14. A system, comprising:
at least one processor; and
memory, the memory storing one or more instructions which, when executed by the at least one processor, cause the at least one processor to:
receive first sensor data associated with a user;
analyze the first sensor data to generate a set of biomarkers;
generate one or more recommendations based, at least in part, upon the generated set of biomarkers;
provide the one or more recommendations for display on a client device;
determine that the user has taken an action with respect to the one or more recommendations;
analyze second sensor data associated with the user; and
cause an indication to be provided for display with respect to the analyzed second sensor data.

15. The system of claim 14, wherein the instructions, when executed by the at least one processor, cause the at least one processor to further:
combine a plurality of biomarkers, of the set of biomarkers, to generate a new biomarker; and
generate the one or more recommendations based, at least in part, upon the generated set of biomarkers and the new biomarker.

16. The system of claim 14, wherein at least one of the first sensor data and the second sensor data is analyzed using machine learning.

17. The system of claim 14, wherein the sensor data is collected from one or more of: light sensors, global positioning system (GPS) sensors, accelerometers, gyroscopes, magnetometers, barometers, network connectivity sensors, activity state sensors, screen touch events, and data from paired wearables.

18. The system of claim 14, wherein the instructions, when executed by the at least one processor, cause the at least one processor to further:
determine a score for at least one biomarker;
determine that the score falls below a determined threshold; and
generate the one or more recommendations based, at least in part, upon the score.

19. The system of claim 14, wherein the instructions, when executed by the at least one processor, cause the at least one processor to further:
determine a baseline score for one or more biomarkers of the set;
determine that a second score for the one or more biomarkers fall below the determined baseline score; and
generate the one or more recommendations based, at least in part, upon the baseline score and the second score.

20. The system of claim 14, wherein the instructions, when executed by the at least one processor, cause the at least one processor to further:
determine a score for at least one biomarker;
generate an alert if the score falls below a determined threshold; and
provide the alert for display on the client device.
